(19) United States
(12) Patent Application Publication
(10) Pub. No.: US 2024/0120106 A1
(43) Pub. Date: Apr. 11, 2024

(54) Interactive bodymap systems and machine learning methods

(71) Applicant: pulseData, Inc., New York, NY (US)

(72) Inventors: Jung Hoon Son, New York, NY (US); Chris Kipers, Pomfret Center, CT (US); Tia Yue Yu, Irvine, CA (US); Teddy Cha, Brooklyn, NY (US); Hai Po Sun, New York, NY (US); Annalee Kuse, New York, NY (US); John Li, Santa Monica, CA (US)

(21) Appl. No.: 18/219,168

(22) Filed: Jul. 7, 2023

Related U.S. Application Data
(60) Provisional application No. 63/367,969, filed on Jul. 8, 2022.

(51) Int. Cl. G16H 50/30; G06F 3/01; G16B 20/00

(52) U.S. Cl. CPC G16H 50/30; G06F 3/011; G06F 3/017; G16B 20/00

(57) ABSTRACT

Methods and systems for displaying a healthcare condition. The methods include receiving at an interface a plurality of inputs of healthcare data associated with a patient, mapping the plurality of inputs of healthcare data to a phenotype using a first ontology system, determining at least one body portion associated with the phenotype using a second ontology system representative of body portions, and generating a first interactive display of the at least one body portion associated with the phenotype.

### CROSS-REFERENCE TO RELATED APPLICATIONS

The present application claims the benefit of and priority to co-pending United States provisional application. No. 63/367,969, filed on Jul. 8, 2022, the entire disclosure of which is incorporated by reference as if set forth in its entirety herein.

### TECHNICAL FIELD

Embodiments described herein generally relate to analyzing healthcare data and, more particularly but not exclusively, to systems and methods for processing healthcare conditions.

### BACKGROUND

The process of providing healthcare to patients produces large amounts of data. As the number of health devices used for monitoring a patient increases in the coming years, the amount of data will further increase.

Existing electronic health record (EHR) interfaces are most suited for inputting data, such as data associated with lab orders, notes, and medication suggestions. However, these existing EHR interfaces may not be as suitable for providing a useful or informative review of a patient or their condition(s). This makes reviewing patient data difficult, and may increase the likelihood of overlooking details associated with the patient's health.

A need exists, therefore, for systems and methods that overcome the disadvantages of existing EHR interfaces.

### SUMMARY

This summary is provided to introduce a selection of concepts in a simplified form that are further described below in the Detailed Description section. This summary is not intended to identify or exclude key features or essential features of the claimed subject matter, nor is it intended to be used as an aid in determining the scope of the claimed subject matter.

In one aspect, embodiments relate to a method for processing a healthcare condition. The method includes receiving at an interface a plurality of inputs of healthcare data associated with a patient; mapping the plurality of inputs of healthcare data to a phenotype using a first ontology system; determining at least one body portion associated with the phenotype using a second ontology system representative of body portions; and generating a first interactive display of the at least one body portion associated with the phenotype.

In some embodiments, the method further includes receiving a user interaction with respect to the first interactive display of the at least one body portion, and transforming the first interactive display to a second interactive display associated with the phenotype. In some embodiments, the user interaction includes a gesture.

In some embodiments, generating the first interactive display of the at least one body portion includes providing a treatment recommendation.

In some embodiments, the first interactive display is generated on an augmented reality display or a virtual reality display.

In some embodiments, the plurality of inputs include data from at least one of electronic health record data, test results, biometrics, or audio inputs.

In some embodiments, generating the first interactive display of the at least one body portion includes displaying at least some of the healthcare data in proximity to the at least one body portion displayed.

In some embodiments, the method further includes autonomously generating a description of the phenotype and presenting the description of the phenotype in the first interactive display.

In some embodiments, the method further includes generating a risk score associated with the phenotype based on the plurality of inputs and at least the first ontology system.

According to another aspect, embodiments relate to a system for processing a healthcare condition. The system includes an interface for receiving a plurality of inputs of healthcare data associated with a patient; and one or more processors executing instructions stored on memory and configured to map the plurality of inputs of healthcare data to a phenotype using a first ontology system, determine at least one body portion associated with the phenotype using a second ontology system representative of body portions, and a first interactive display configured to present the at least one body portion associated with the phenotype.

In some embodiments, the first interactive display is further configured to receive a user interaction with respect to the first interactive display of the at least one body portion, and transform the first interactive display to a second interactive display associated with the phenotype. In some embodiments, the user interaction includes a gesture.

In some embodiments, the first interactive display provides a treatment recommendation.

In some embodiments, the first interactive display is generated on an augmented reality display or a virtual reality display.

In some embodiments, the plurality of inputs include data from at least one of electronic health record data, test results, biometrics, or audio inputs.

In some embodiments, the first interactive display of the at least one body portion includes at least some of the healthcare data in proximity to the at least one body portion displayed.

In some embodiments, the one or more processors are further configured to autonomously generate a description of the phenotype, and the first interactive display is configured to present the description of the phenotype.

In some embodiments, the one or more processors are further configured to generate a risk score associated with the phenotype based on the plurality of inputs and at least the first ontology system.

According to another aspect, embodiments relate to a computer program product for processing a healthcare condition, the computer program product comprising computer executable code embodied in one or more non-transitory computer readable media. The executable code, when executing on one or more processors, performs the steps of receiving at an interface a plurality of inputs of healthcare data associated with a patient, mapping the plurality of inputs of healthcare data to a phenotype using a first ontology system, determining at least one body portion associated with the phenotype using a second ontology system representative of body portions, and generating a first interactive display of the at least one body portion associated with the phenotype.

In some embodiments, the computer program product further includes computer executable code that, when executing on one or more processors, performs the steps of receiving a user interaction with respect to the first interactive display of the at least one body portion and transforming the first interactive display to a second interactive display associated with the phenotype.

### BRIEF DESCRIPTION OF DRAWINGS

Non-limiting and non-exhaustive embodiments of this disclosure are described with reference to the following figures, wherein like reference numerals refer to like parts throughout the various views unless otherwise specified.

FIG. 1 illustrates a system for processing a healthcare condition in accordance with one embodiment;

FIG. 2 presents a sigmoid function for classifying or predicting a phenotype in accordance with one embodiment;

FIGS. 3A & 3B illustrate a support vector machine model for classifying or predicting a phenotype in accordance with one embodiment;

FIG. 4 illustrates a random forest decision tree model for classifying or predicting a phenotype in accordance with one embodiment;

FIG. 5 illustrates a display of a patient's medical conditions in accordance with one embodiment;

FIGS. 6A-C illustrate various displays of a patient's healthcare data in accordance with one embodiment;

FIG. 7 illustrates a display of a patient's medical conditions in accordance with another embodiment;

FIG. 8 depicts a display of text depictions of ontology mappings in accordance with one embodiment; and

FIG. 9 depicts a flowchart of a method for processing a healthcare condition in accordance with one embodiment.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment. The appearances of the phrase “in some embodiments” in various places in the specification are not necessarily all referring to the same embodiments.

Some portions of the description that follow are presented in terms of symbolic representations of operations on non-transient signals stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

The embodiments described herein provide novel techniques for analyzing patient data and processing healthcare conditions. These offer improved techniques for displaying healthcare data, which has become increasingly complex due at least in part to the constantly-increasing number of health monitoring devices.

The embodiments herein leverage one or more ontologies to infer relationships between healthcare data features associated with a patient. As described in the present application, the term “feature” may refer to a healthcare data attribute or a value thereof. One or more displays may then present this information in a way that is meaningful to a viewer. For example, the embodiments herein may present data in one or more display types that allow a viewer such as a healthcare provider to understand the relationship between different aspects of a patient's health. This may give the user a comprehensive view or understanding of the patient's condition(s) or overall health.

For example, a patient may want to know which organs or body parts are affected by a disease. Medical personnel may want to know whether any conditions affect a patient's kidneys so they will endeavor to avoid nephrotoxic medications. As another example, a researcher may want to explore which cellular pathways and organs may be affected by unknown genomic variants based on existing knowledge of a gene product's function.

In operation, the embodiments herein may computationally map healthcare data to a phenotype using a terminology or ontology for a disease, condition, or physiological state. The abstracted phenotype(s) may then be algorithmically evaluated to determine which body part, organ, system, or other bodily component(s) are associated with the phenotype. For example, the embodiments herein may rely on or otherwise leverage the International Classification of Disease (“ICD”) ontology, maintained by the World Health Organization and available at https://www.who.int/standards/classifications/classification-of-diseases. The ICD is a diagnostic tool and provides knowledge on the causes, extent, and consequences of diseases, and designates phenotypes by codes.

Accordingly, the embodiments herein may map received healthcare data to a phenotype using a standard terminology or ontology for disease, condition, or physiological state, which itself would embody one or more phenotypes. For example, code I50.9 of the ICD is mapped to the condition of “heart failure.” Additionally or alternatively, the embodiments herein may identify a condition based only on patient data. For example, a hemoglobin count A1c>9.0 may suggest that a patient has diabetes.

The systems and methods herein may then use the determined conditions and their associated phenotypes to determine which body part, organ, systems, or combination thereof are affected. For example, the mapped condition of “heart failure” may be evaluated as a condition that affects the heart, the cardiovascular system, or both. Similarly, “type 1 diabetes” may be mapped to one or more of the endocrine system, pancreas, pancreatic islet cells, beta cells, or the like.

The systems and methods described herein may perform this mapping by leveraging and extracting knowledge mappings across ontologies or terminologies to generate differing levels of knowledge for a provided context. For example, one such pipeline may link ICD codes to the Disease Ontology (https://disease-ontology.org/), the Human Phenotype Ontology (https://hpo.jax.org/app/), and the MONDO Ontology (https://mondo.monarchinitiative.org/). The embodiments herein may use the relationships stored within these ontologies to map healthcare data to one or more phenotypes using an ontology system that represents human body parts.

Having determined which body parts, organs, or organ systems are affected by the mapped condition(s), the embodiments herein may then display the body parts, organs, organ systems, and relevant data superimposed on imagery such as a rendering of a human body or portion thereof. The display may be an augmented reality display, a virtual reality display, or the like.

Regardless of the exact type of display or presentation, the embodiments herein may include or otherwise provide an interactive interface that responds to user inputs to control the presented information. A user may provide inputs such as mouse cursor events or visual indicia such as gestures (e.g., through movement of their hands, fingers, eyes, etc.). These inputs may adjust the level of details presented, such as by expanding the pathology and physiology of an affected body part, organ, organ system, tissue, cell, or the like.

The interactive displays may also present different levels of detail based on the user and their needs. For patients, the affected organ such as “kidney” will suffice. However, medical personnel such as specialist physicians may require a review of which part of the glomerular basement membrane of the kidney's glomeruli are affected by a given phenotype.

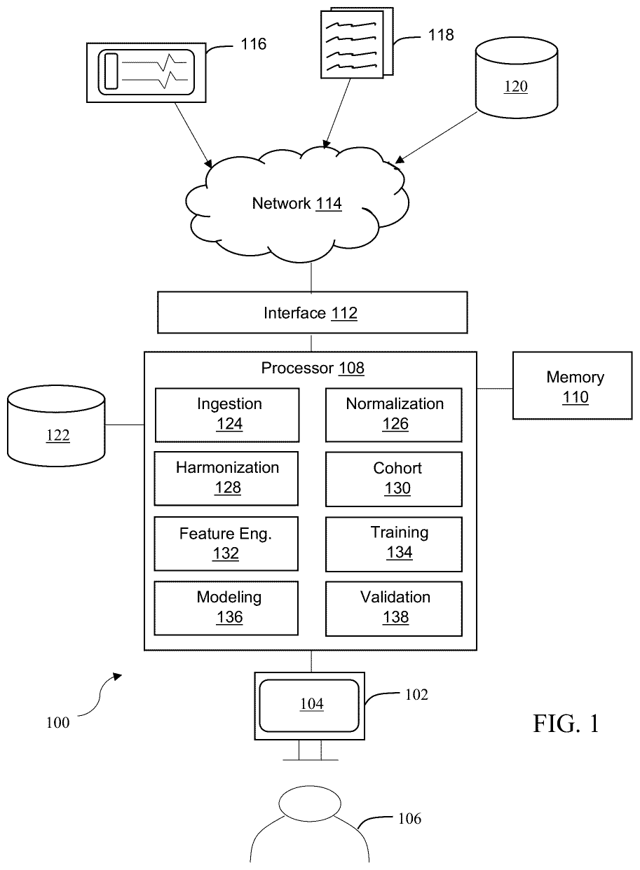

FIG. 1 illustrates a system 100 for processing a healthcare condition in accordance with one embodiment. The system 100 may include a user device 102 executing a user interface 104 accessible by a user 106. The user 106 may include healthcare personnel such as a treating clinician, for example.

The user device 102 may be any sort of device that can visually present information. For example, in some embodiments, the user device may be a personal computer (PC), laptop, tablet, smartphone, television, smartwatch, virtual- or augmented-reality headsets, etc. This list of devices is exemplary, and other devices whether available now or invented hereafter may be used in conjunction with the embodiments herein.

The user device 102 may be in operable connectivity with one or more processors 108 executing instructions stored on memory 110. The processor(s) 108 may be any hardware device capable of executing instructions stored on memory 110 to provide various components or modules. The processor 108 may include a microprocessor, a field programmable gate array (FPGA), an application-specific integrated circuit (ASIC), or other similar devices.

In some embodiments, such as those relying on one or more ASICs, the functionality described as being provided in part via software may instead be configured into the design of the ASICs and, as such, the associated software may be omitted. The processor 108 may be configured as part of the user device 102 (e.g., a laptop) or located at some remote location.

The memory 110 may be L1, L2, L3 cache, or RAM memory configurations. The memory 110 may include non-volatile memory such as flash memory, EPROM, EEPROM, ROM, and PROM, or volatile memory such as static or dynamic RAM, as discussed above. The exact configuration/type of memory 110 may of course vary as long as instructions for processing a healthcare condition can be performed by the system 100.

The system 100 may also include an interface 112 to receive healthcare data over one or more networks 114. The network(s) 114 may link the various components with various types of network connections. The network(s) 114 may be comprised of, or may interface to, any one or more of the Internet, an intranet, a Personal Area Network (PAN), a Local Area Network (LAN), a Wide Area Network (WAN), a Metropolitan Area Network (MAN), a storage area network (SAN), a frame relay connection, an Advanced Intelligent Network (AIN) connection, a synchronous optical network (SONET) connection, a digital T1, T3, E1, or E3 line, a Digital Data Service (DDS) connection, a Digital Subscriber Line (DSL) connection, an Ethernet connection, an Integrated Services Digital Network (ISDN) line, a dial-up port such as a V.90, a V.34, or a V.34bis analog modem connection, a cable modem, an Asynchronous Transfer Mode (ATM) connection, a Fiber Distributed Data Interface (FDDI) connection, a Copper Distributed Data Interface (CDDI) connection, or an optical/DWDM network.

The network or networks 114 may also comprise, include, or interface to any one or more of a Wireless Application Protocol (WAP) link, a Wi-Fi link, a microwave link, a General Packet Radio Service (GPRS) link, a Global System for Mobile Communication (GSM) link, a Code Division Multiple Access (CDMA) link, or a Time Division Multiple access (TDMA) link such as a cellular phone channel, a Global Positioning System (GPS) link, a cellular digital packet data (CDPD) link, a Research in Motion, Limited (RIM) duplex paging type device, a Bluetooth radio link, or an IEEE 802.11-based link.

The interface 112 may receive healthcare data from a variety of data sources over the network(s) 114. This healthcare data may relate to or otherwise be obtained through treatment of a patient. The sources of the healthcare data may include, but are not limited, patient monitoring devices 116, clinical notes 118 (whether made electronically or on paper), databases 120, or the like.

The processor(s) 108 may also be in communication with or otherwise have access to one or more ontology databases 122. The ontology databases 122 may store publicly available medical ontologies or data associated therewith. These provide universally-accepted standards for identifying medical laboratory observations, coding systems, and terminologies.

For example, the ontology databases 122 may store data regarding the Uberon Ontology (https://obofoundry.org/ontology/uberon.html), Disease Ontology, Human Phenotype Ontology, MONDO Ontology, or any other type of ontology whether available now or established hereafter. Additionally, the embodiments herein may leverage a Relations Ontology (https://obofoundry.org/ontology/ro.html) to connect data across different ontologies. The ontology databases 122 may also include any custom-generated ontologies.

The one or more processor(s) 108 may include or otherwise execute one or more modules for analyzing healthcare data, modeling the healthcare data, and visually presenting data associated with the healthcare data. These modules may include, but are not limited to, a data ingestion module 124, a normalization module 126, a harmonization module 128, a cohort creation module 130, a feature engineering module 132, a training module 134, a modeling module 136, and a validation module 138.

In operation, the ingestion module 124 may receive from the interface 112 healthcare data related to a patient. For example, the healthcare data may include, but is not limited to, lab test results, billing data, clinical laboratory medication history, pharmacy data, biometrics, vitals, clinical notes, audio or textual inputs, data generated from electronic health records (EHR) systems, or the like. In some embodiments, the ingestion module 124 may receive this data as part of a training phase for generating supervised machine learning models.

As the healthcare data may be received from several different sources, the healthcare data may include data in a variety of formats. For example, and without limitation, the healthcare data may include text files, audio files, CSV files, digital data, or the like.

The normalization module 126 may execute instructions stored on memory 110 to perform one or more procedures to normalize the received healthcare data. This ensures that all data is represented in the same format. For example, data represented in units of “lbs,” “LBs,” and “pounds” may be converted to “lbs.” Similarly, the memory 110 may store conversion factors between units to enable the normalization module 126 to execute any appropriate unit conversion procedures (e.g., to convert kilograms to pounds).

The harmonization module 128 may execute instructions stored on memory 110 to ensure all data is on the same scale. For example, if healthcare data associated with a patient includes an “age” attribute with an associated value of “0” (zero), and a “weight” attribute with a value of “3,000” the meaning of these values may not be readily apparent to a user. However, an age value of “0” may suggest that the patient is a newborn child. In this scenario, the harmonization module 128 may recognize that a weight value of, e.g., 2,500 to 5,000 may be in grams as those are typical weights of newborn children. The harmonization module 128 may then perform any unit conversion procedures to, for example and if necessary, convert the patient's weight to lbs.

The cohort module 130 may execute one or more processes to define conditions the system 100 will predict or otherwise identify based on the received healthcare data. In other words, the cohort module 130 may execute one or more cohort procedures for converting a patient dataset to an event dataset. For example, the cohort module 130 may create groups or otherwise definitions for certain conditions. These may be based on data from the one or more ontologies in the ontology database 122. In some embodiments, a cohort may specify a disease, symptoms of the disease, affected body parts, recommendations for treatment, or the like.

As an example, a user may be interested in an end-of-month health metric for one hundred (100) patients. The user may have monthly EHR records for an entire calendar year. The cohort module 130 may expand the population dataset (e.g., to Patient 1, Patient 2 . . . Patient 100) to a desired event dataset (e.g., with entries of Patient 1 January, Patient 1 February, etc.). Accordingly, the cohort module 130 would convert the Patient dataset of 100 entries to an Event dataset of 1,200 entries. This may be particularly important for smaller datasets in which the number of patients may limit the quality of results.

More specifically, cohorts may be further tailored for certain age groups, classes, geographic locations of patients, or the like. For example, there a may be a cohort for diabetes directed towards patients aged 0-18 years old, a cohort for patients aged 18-40 years old, and a cohort for patients older than 40 years old.

The feature engineering module 132 may identify the variables or combination of variables that are likely predictive of a condition. For example, the feature engineering module 132 may perform one or more procedures involving identifying, extracting, transforming, or otherwise manipulating raw data to select the feature(s) that can most accurately predict a condition.

In some embodiments, the feature engineering process may first involve a user (e.g., a data scientist, medical personnel, etc.) providing input regarding one or more variables. For example, medical personnel may first indicate which features tend to be predictive of certain phenotypes. Additionally or alternatively, the ontology databases 122 may store data regarding the relationship between features and phenotypes.

The feature engineering module 132 may also perform any required transformation procedures on the selected data, in addition to or in lieu of procedures performed by the harmonization module 128. For example, the feature engineering module 132 may ensure features or variables are on the same scale and also eliminate outliers to ensure analyzed features are within an acceptable range. The feature engineering module 132 may use techniques based on principal component analysis, linear discriminate analysis, clustering, one-hot encoding, multi-relationship decision tree learning, or the like.

The training module 134 may segregate the healthcare data into a subset upon which model development may occur. This allows a user to retain a test subset for testing and validating the generated models, as discussed below with respect to the validation module 138.

The modeling module 136 may use machine learning techniques to identify statistical models that are significant. The embodiments herein may execute any one or more of a variety of different types of machine learning models. Generally, in operation, these models take as input one or more features of healthcare data associated with a patient, and output some classification or prediction. For example, these predictions or classifications may refer to a condition also affecting the patient (or likely to affect the patient), even if the condition is not in the patient record.

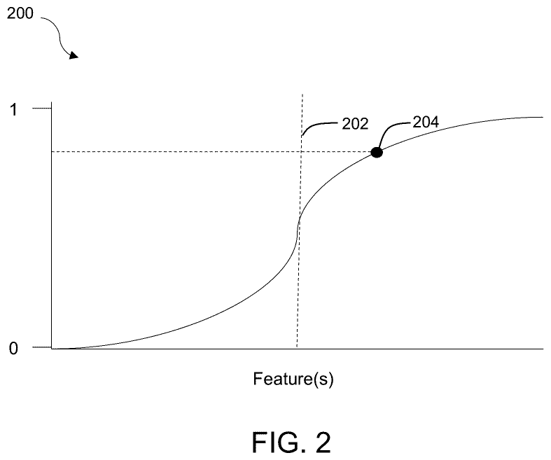

For example, FIG. 2 illustrates a graphical representation of a sigmoid function 200 in accordance with one embodiment. The sigmoid function 200 may classify a patient as having a condition or not having a condition, depending on associated feature(s). That is, the sigmoid function 200 may represent the probability that a particular patient, based on healthcare data features on the x-axis, has a particular condition. The x-axis may correspond to, for example, lab results, symptoms, diagnosed conditions, or the like.

A user such as medical personnel may specify a decision boundary 202 such that feature(s) that place the patient to the right of the boundary 202 have a higher probability of being associated with a particular phenotype than not being associated with the phenotype. Accordingly, healthcare data that places features to the right of the boundary 202 may be classified as being associated with a particular phenotype, and healthcare data that places features to the left of the boundary 202 may be classified as not being associated with a particular phenotype.

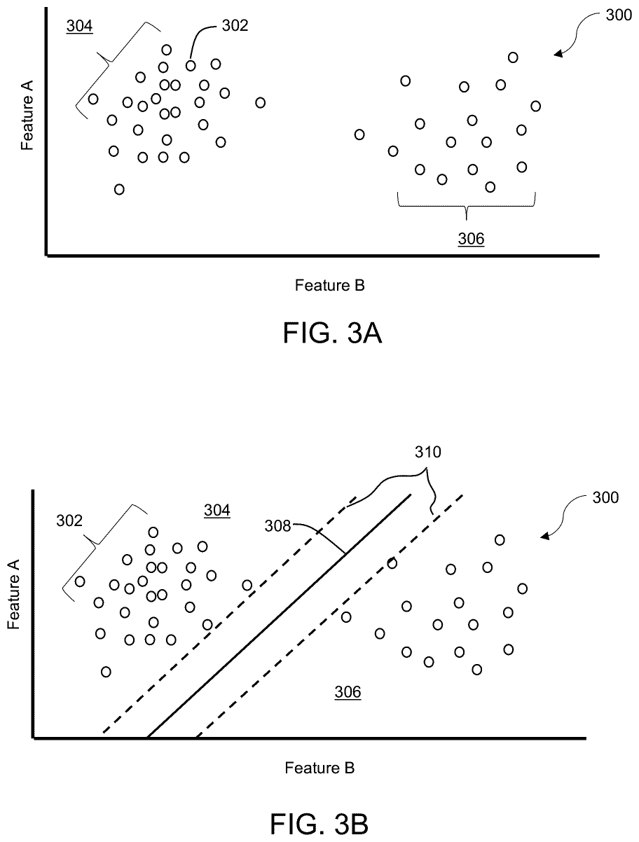

As another example, the modeling module 136 may execute a support vector machine (SVM) model to classify or predict a phenotype based on patient healthcare data. FIG. 3A, for example, illustrates an SVM plot 300 in accordance with one embodiment. The plot 300 illustrates a plurality of support vectors 302, illustrated as circles, representing feature vectors that are either labeled as being associated with a particular phenotype in group 304 (on the left side of the plot 300), or feature vectors that are labeled as not being associated with the particular phenotype in group 306 (on the right side of the plot 300).

The modeling module 136 may determine an appropriate decision boundary such that features falling on the left side thereof will predict or classify the patient as having a particular phenotype, and features falling on the right side of the boundary 308 will be predict or classify the patient as not having the phenotype. FIG. 3B illustrates a generated decision boundary 308 with margins 310. Though SVM training, a preferred decision boundary 308 can be discovered such that the margins 310 are as large as possible for a given set of features.

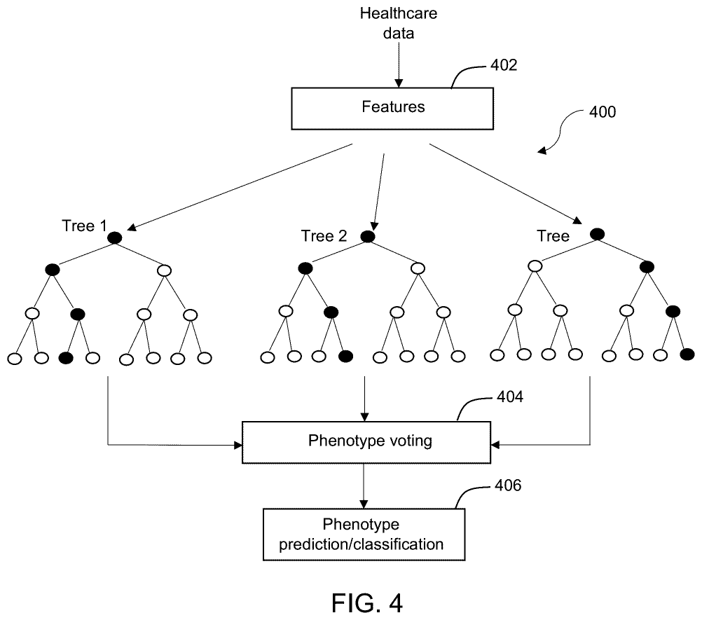

As yet another example, the modeling module 134 may execute one or more random forest models to classify or predict a phenotype based on patient healthcare data. FIG. 4, for example, illustrates a random forest classifier 400 that receives as input healthcare features 402 related to a patient. The random forest classifier 400 may include a plurality of trees that are each configured to detect whether some feature(s) are present or are otherwise associated with a patient.

For example, Tree 1 may determine whether the patient is diabetic. If so, the classifier 400 may traverse a certain branch of the tree, which may then consider whether some other feature is present. FIG. 4 illustrates certain nodes of Trees 1-3 darkened, indicating the presence or absence of some feature. Although only three trees are illustrated in FIG. 4, any number of trees may be used to consider any number of features.

Each tree may output a classification decision or prediction regarding whether a patient has a certain condition or disease. These outputs may be based on the presence or absence of certain features, represented by the darkened notes in Trees 1-3.

The classification decisions of the trees may be combined in step 404, and a prediction is provided in step 406. The classifier 400 may predict that a patient has a certain disease if, for example, the majority of the trees output a classification decision in step 404 that suggest the patient has the disease.

The modeling module 134 may also output a risk score associated with a predicted phenotype. For example, the risk score may represent the probability that a patient has a predicted phenotype, or may represent the severity of the predicted phenotype. The risk score may be based on the number of features present in the patient healthcare data, the number of models outputting a positive prediction, or some combination thereof.

Referring back to FIG. 1, the validation module 138 may review the statistical models to assess their performance. The validation module 138 may use a holdout data set to evaluate the performance of the generated model(s). In these cases, the holdout dataset may include data that was not used to generate the model(s), but may include similar data features. In some embodiments, the holdout data may include the most recently-gathered healthcare data.

In some embodiments, the validation module 138 may rely on k-fold, cross validation techniques for generating and assessing models. In these techniques, the dataset is partitioned into k sections, and k−1 of the sections are used to train a model. The remaining section that was not used to generate the model is used as the holdout dataset. The generated model may then analyze the holdout dataset and make classifications or predictions based on the analysis.

For example, for each analyzed model, the validation module 138 may compute one or more performance metrics such as the number of true positive classifications, the number of false positive classifications, the number of true negative classifications, the number of false negative classifications, F1 score, accuracy, precision, recall, specificity, the precision-recall curve, the Receiver Operating Characteristics curve, or the like.

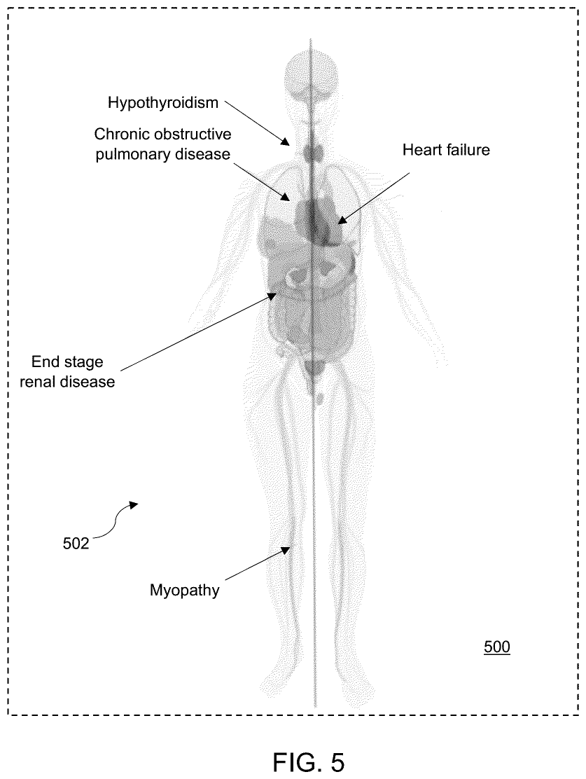

As discussed previously, upon making predictions or classifying healthcare data, the systems and methods described herein visually present data using novel techniques to offer advantages over existing display methodologies. For example, FIG. 5 illustrates a display 500 of a patient's medical conditions on a stylized rendering of a patient's body 500 in accordance with one embodiment. The display 500 may be presented on a 2-dimensional display (such on a laptop, PC, tablet), a 3-dimensional display, a holograph, a virtual reality display, an augmented reality display, or some combination thereof.

As illustrated, the patient's EHR data processed as described above indicates that the patient suffers from several conditions, including heart failure. The system 100 has inferred the body part associated with each phenotype, and has displayed it in proximity to a stylized display of the body part. For example, heart failure is displayed with a callout pointing to the heart, hypothyroidism is displayed with a callout to the thyroid, renal disease is displayed with a callout to the kidney, etc.

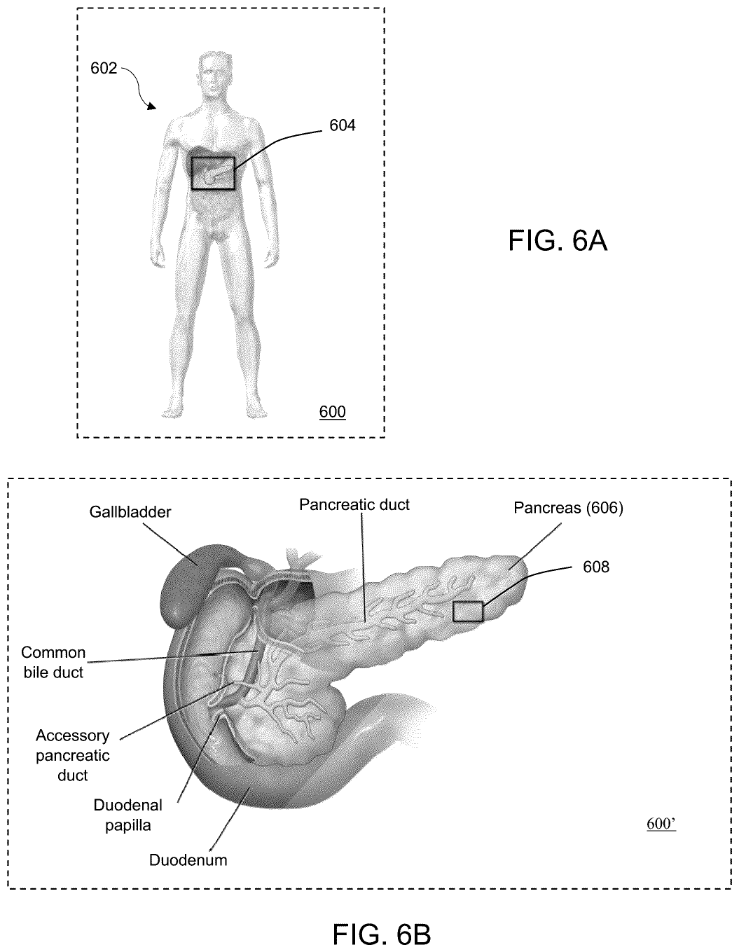

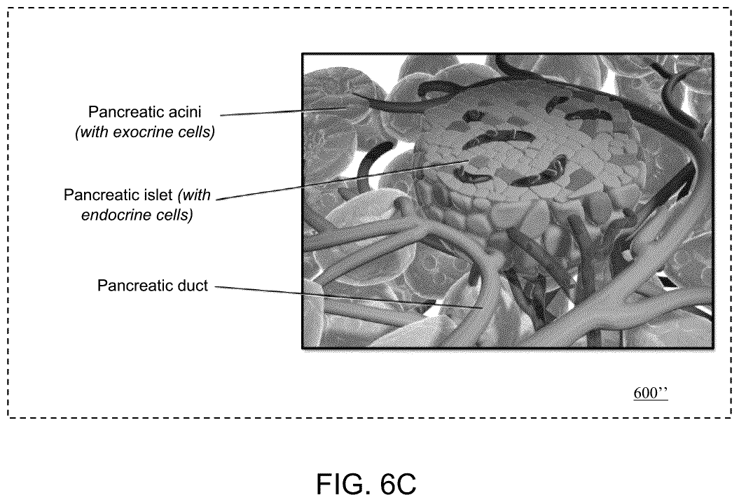

FIGS. 6A-C illustrate how a particular body part, component, or organ may be selected and viewed in accordance with one embodiment. For example, FIG. 6A presents a display 600 showing various aspects of a patient's body 600 in accordance with one embodiment. For example, the display 600 of the patient's body 602 may correspond to the patient of FIG. 5 (and may have the same ailments). A user such as a treating clinician may select a callout (illustrated as a box) 604 to view details associated with the patient's pancreas. For example, the user may use a cursor to select a certain area on which to zoom in, use a gesture, or activate a portion of a touch-screen device to create the callout 604.

As a result of the user input in FIG. 6A, FIG. 6B may present a display 600′ of a detailed view of the user's pancreas 606. Similar to FIG. 6A, FIG. 6B shows callouts highlighting certain components of the pancreas. The user may further select a callout (illustrated as a box) 608 to view details associated with a region of the patient's pancreas 606.

Similar to FIGS. 6A & 6B, FIG. 6C shows callouts highlighting certain components of the pancreas associated with callout 608. For example, the zoomed-in display 600″ of FIG. 6C may show that a patient with Type I diabetes suffers from depleted islet cells based on insulin levels in the patient's EHR.

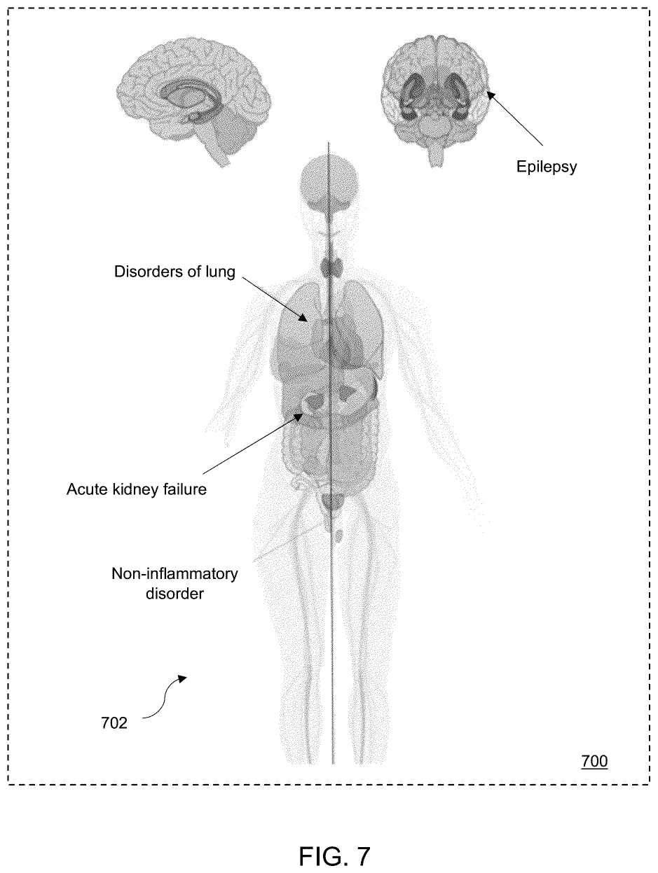

FIG. 7 illustrates a display 700 in accordance with another embodiment. The display includes imagery of a patient's body 702, and may also include various callouts highlighting body organs and associated phenotypes. The data associated with these callouts may be obtained from one or more ontologies. For example, the received healthcare data and the analysis thereof by one or more machine models may indicate a patient suffers from kidney disease and low Estimated Glomerular Filtration Rate (eGFR). Accordingly, the display 700 may highlight the patient's kidneys and upper urinary system. A user such as a treating clinician may provide some input with respect to the kidney on the display, such as by clicking on the kidney or creating a callout or box with respect to the kidney as discussed previously. The display 700 may then zoom in on the patient's glomuleri, which may be presented in a certain color or with other indicia to show low filtration rate.

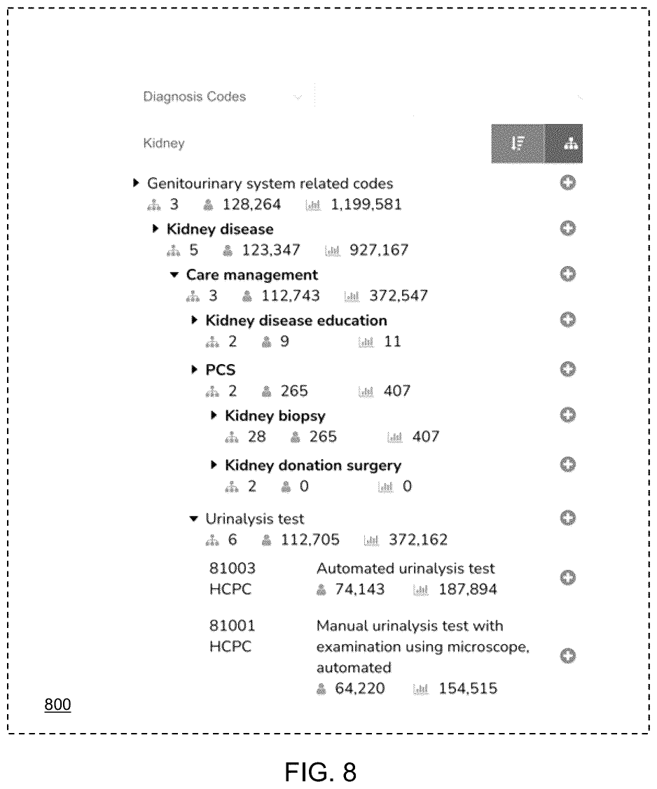

In some embodiments, the systems and methods herein may also provide a user with accompanying text describing ontology mapping and predicted phenotypes. For example, FIG. 8 depicts a display 800 of text depiction of ontology mappings across various procedure codes, diagnosis codes, lab results, and EHR data. These are mapped across multiple conceptual frameworks. As seen in FIG. 8, these may include (1) Body System (genitourinary), (2) Disease/condition (kidney disease), (3) Treatment (education on kidneys), (4) Related lab tests (urinalysis), and (5) Related biological factors (e.g., proteins that cause renal fibrosis). Accordingly, a user such as a treating clinician may view and share various types of data regarding a phenotype to obtain a more comprehensive view of a patient's health.

A user may leverage the data of display 800 in a variety of ways and, specifically, to link different data sources to a patient and organ. For example, a user may use these different frameworks to target different users or to view different types of data that is linked to the same physical body part. Similarly, a care manager may view the presented data to learn about patient education and related conditions, and a nephrologist or other type of treating clinician may want to understand biological factors such as the patient's eGFR. Similarly, a patient may want a general understanding of the disease or their organ(s).

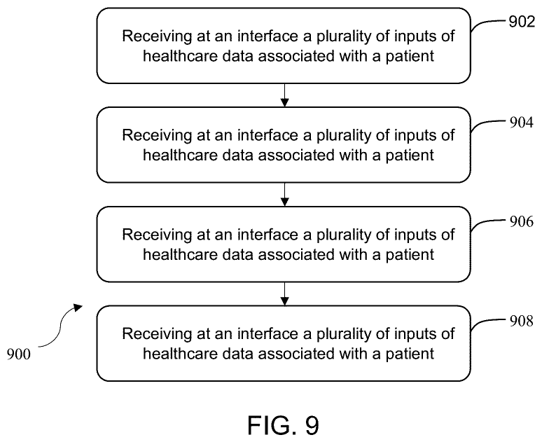

FIG. 9 depicts a flowchart of a method 900 for visually displaying healthcare data in accordance with one embodiment. The system of FIG. 1 or the components thereof may perform one or more of the steps of method 900.

Step 902 involves receiving at an interface a plurality of inputs of healthcare data associated with a patient. For example, the healthcare data may be received over one or more networks and from one or more sources. The healthcare data may include data obtained from vital sign measurements, lab results, billing data, a patient's EHR, or the like.

Step 904 involves mapping the plurality of inputs of healthcare data to a phenotype using a first ontology system. The embodiments herein may execute one or more machine learning models to classify or otherwise predict a patient condition based on the received healthcare data. These models may include supervised machine learning models trained or otherwise generated through the series of steps discussed previously, and may leverage publicly-accessible or custom ontologies.

Step 906 involves determining at least one body portion associated with the phenotype using a second ontology system representative of body portions. As discussed previously, the embodiments herein may reference one or more additional ontologies based on the mapped phenotype from the first ontology to identify a one or more body portions (e.g., organs) also affected. In some embodiments, the identified body portion may be affected by a condition that is not in the patient's record.

Step 908 involves generating a first interactive display of the at least one body portion associated with the phenotype. The display may be interactive in that it allows a user (e.g., medical personnel, a patient, etc.) to view imagery associated with the patient's medical condition, and also provide some input to control what information is presented and how the information is presented. For example, and as discussed previously, a user may provide some input such as a cursor action or a gesture to highlight certain organs on a display to obtain a more detailed view of the patient.

The term “tangible computer-readable storage medium” shall accordingly be taken to include, but not be limited to: solid-state memories such as a memory card or other package that houses one or more read-only (non-volatile) memories, random access memories, or other re-writable (volatile) memories, a magneto-optical or optical medium such as a disk or tape, or other tangible media which can be used to store information. Accordingly, the disclosure is considered to include any one or more of a tangible computer-readable storage medium, as listed herein and including art-recognized equivalents and successor media, in which the software implementations herein are stored.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.

What is claimed is:

1. A method for processing a healthcare condition, the method comprising:
receiving at an interface a plurality of inputs of healthcare data associated with a patient;
mapping the plurality of inputs of healthcare data to a phenotype using a first ontology system;
determining at least one body portion associated with the phenotype using a second ontology system representative of body portions; and
generating a first interactive display of the at least one body portion associated with the phenotype.

2. The method of claim 1 further comprising:
receiving a user interaction with respect to the first interactive display of the at least one body portion, and
transforming the first interactive display to a second interactive display associated with the phenotype.

3. The method of claim 2 wherein the user interaction includes a gesture.

4. The method of claim 1 wherein generating the first interactive display of the at least one body portion includes providing a treatment recommendation.

5. The method of claim 1 wherein the first interactive display is generated on an augmented reality display or a virtual reality display.

6. The method of claim 1 wherein the plurality of inputs include data from at least one of electronic health record data, test results, biometrics, or audio inputs.

7. The method of claim 1 wherein generating the first interactive display of the at least one body portion includes displaying at least some of the healthcare data in proximity to the at least one body portion displayed.

8. The method of claim 1 further comprising:
autonomously generating a description of the phenotype, and
presenting the description of the phenotype in the first interactive display.

9. The method of claim 1 further comprising generating a risk score associated with the phenotype based on the plurality of inputs and at least the first ontology system.

10. A system for processing a healthcare condition, the system comprising:
an interface for receiving a plurality of inputs of healthcare data associated with a patient;
one or more processors executing instructions stored on memory and configured to:
map the plurality of inputs of healthcare data to a phenotype using a first ontology system,
determine at least one body portion associated with the phenotype using a second ontology system representative of body portions, and
a first interactive display configured to present the at least one body portion associated with the phenotype.

11. The system of claim 10 wherein the first interactive display is further configured to:
receive a user interaction with respect to the first interactive display of the at least one body portion, and
transform the first interactive display to a second interactive display associated with the phenotype.

12. The system of claim 11 wherein the user interaction includes a gesture.

13. The system of claim 10 wherein the first interactive display provides a treatment recommendation.

14. The system of claim 10 wherein the first interactive display is generated on an augmented reality display or a virtual reality display.

15. The system of claim 10 wherein the plurality of inputs include data from at least one of electronic health record data, test results, biometrics, or audio inputs.

16. The system of claim 10 wherein the first interactive display of the at least one body portion includes at least some of the healthcare data in proximity to the at least one body portion displayed.

17. The system of claim 10 wherein the one or more processors are further configured to autonomously generate a description of the phenotype, and the first interactive display is configured to present the description of the phenotype.

18. The system of claim 10 wherein the one or more processors are further configured to generate a risk score associated with the phenotype based on the plurality of inputs and at least the first ontology system.

19. A computer program product for processing a healthcare condition, the computer program product comprising computer executable code embodied in one or more non-transitory computer readable media that, when executing on one or more processors, performs the steps of:
receiving at an interface a plurality of inputs of healthcare data associated with a patient;
mapping the plurality of inputs of healthcare data to a phenotype using a first ontology system;
determining at least one body portion associated with the phenotype using a second ontology system representative of body portions; and
generating a first interactive display of the at least one body portion associated with the phenotype.

20. The computer program product of claim 19 wherein the computer program product further comprises computer executable code that, when executing on one or more processors, performs the steps of receiving a user interaction with respect to the first interactive display of the at least one body portion and transforming the first interactive display to a second interactive display associated with the phenotype.
